(19) United States
(12) Plant Patent Application Publication
(10) Pub. No.: US 2016/0113173 P1
(43) Pub. Date: Apr. 21, 2016

(54) CITRUS RETICULATA PLANT NAMED 'C37'

(50) ;

(71) Applicant: Bill Roe, Eagle Lake, FL (US)

(72) Inventor: Bill Roe, Eagle Lake, FL (US)

(21) Appl. No.: 14/121,775

(22) Filed: Oct. 15, 2014

(51) Int. Cl. A01H 5/00

(52) U.S. Cl. PLT/201

(57) ABSTRACT

A new and distinct Citrus reticulata cultivar named ‘C37’ is disclosed, characterized by production of large, nearly seedless fruit of beautiful internal and external color that peels very easy. Plants to not require a cross-pollinator for fruit production. The new cultivar is a Citrus reticulata, useful for commercial fruit production.

### LATIN NAME OF THE GENUS AND SPECIES

Citrus reticulata

### VARIETY DENOMINATION

‘C37’

### BACKGROUND OF THE INVENTION

The new cultivar is a product of a planned breeding program conducted during 2000 under the direction of the inventor, Bill Roe, a citizen of the United States. The inventor no longer has record of the seed parent, the pollen parent is assumed to be the unpatented variety Citrus reticulata ‘Honey Tang’.

This new variety was found in November 2006 in a commercial nursery in Winter Haven, Fla. Plants were first propagated by tissue culture in 2006 and subsequently grafted onto rootstocks US-897 and US-942. Development, evaluation, tissue culture and grafting all first took place at the inventor's commercial nursery in Winter Haven, Fla. USA. Subsequent evaluations of the variety have shown the characteristics to be true to type.

### SUMMARY OF THE INVENTION

The cultivar ‘C37’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘C37’. These characteristics in combination distinguish ‘C37’ as a new and distinct Citrus reticulata cultivar:

1. Large fruit with Moderately slick skin fruit.

2. Low seed count.

3. Very easy and dry to peel.

4. High brix and moderate acid fruit with delightful flavor.

5. Self pollinating.

6. Heavy fruiting.

7. Has a crunchy and yet juicy internal disposition with a rich juice color.

### COMPARISON TO PARENT VARIEITES

Seed Parent is unknown. Plants of the new cultivar ‘C37’ are similar to plants of the pollen parent, Citrus reticulata ‘Honey Tang’ in most horticultural characteristics, however, plants of the new cultivar ‘C37’ differ in the following ways;

1. Fruit is much earlier to mature.

2. Peel color a much deeper orange.

3. Significantly fewer seeds.

4. Less oblate fruit shape.

5. Tree grows less upright.

### COMMERCIAL COMPARISON

The new variety is best compared to the commercial variety, ‘Tangelo,’ unpatented. ‘C37’ is similar to ‘Tangelo’ in many horticultural characteristics; however, ‘C37’ differs in the following;

1. Much easier to peel.

2. Peels dry with very little peel oil.

3. Fruit less sensitive to plugging.

4. Internal fruit color is a deeper orange.

5. Far fewer seeds.

### BRIEF DESCRIPTION OF THE PHOTOGRAPH

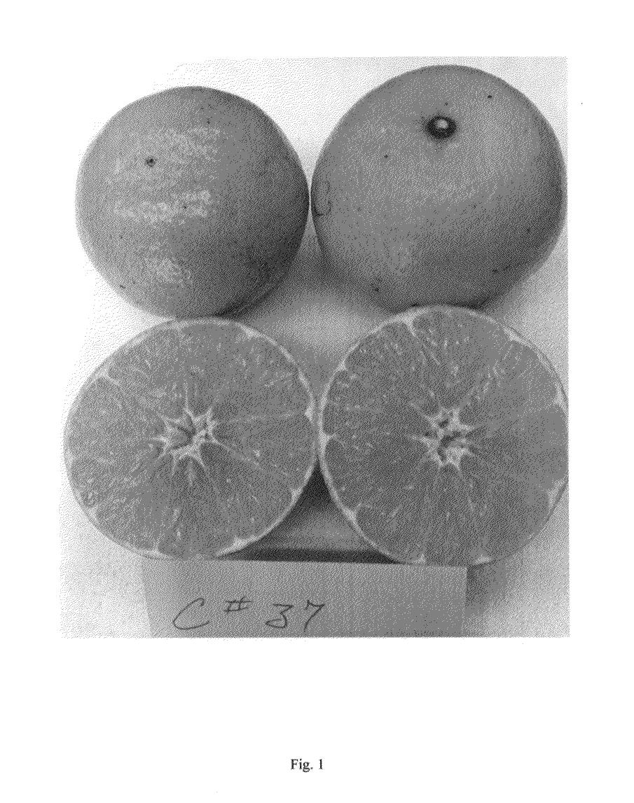

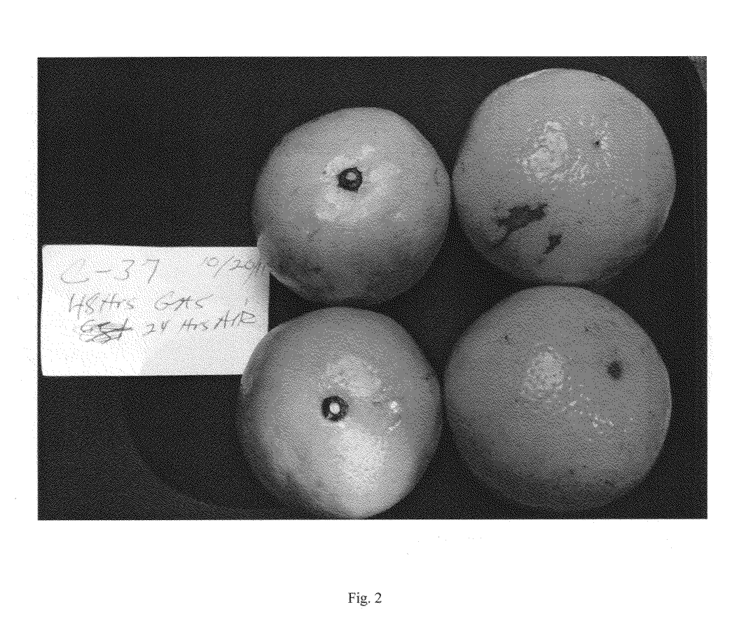

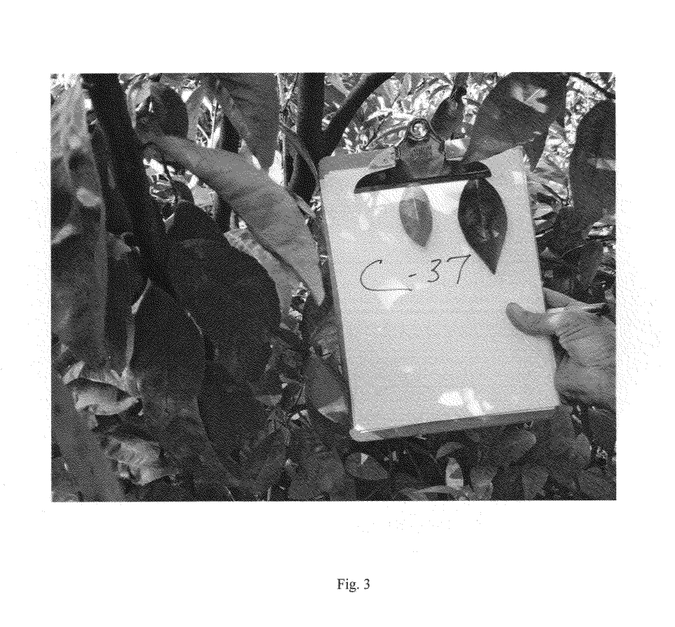

The accompanying photograph in FIG. 1 illustrates in full color examples of typical fruit harvested from ‘C37’. FIG. 2 illustrates in full color the exterior of the fruit, before and after treatment with ethylene gas. FIG. 3 illustrates branches and typical foliage of ‘C37’. Photographs are taken of plants approximately 2 years old. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘C37’ plants grown outdoors under commercial trial conditions in Winter Haven, Fla. USA. The growing temperature ranged from 10° C. to 35° C. during the day and −4° C. to 20° C. at night. Measurements and numerical values represent an average from the most typical specimens.
Botanical classification: Citrus reticulata ‘C37’
Propagation: ‘C37’ is typically grafted onto rootstock of Citrus reticulata US-897 and US-942
Plant:
Age of the plant described: Approximately 2 years.
Plant habit: Below average canopy density.
Height: Approximately 185 cm.
Growth rate: Approximately 2 years to 185 cm.
Plant spread: Wider than average row width, as the tree grows a lot of fruit on the extremity.
Vigor: Good
Length of primary lateral branches: Approximately 40-70 cm. Average of 60 cm
Quantity of lateral branches: 2-4, Average of 4
Angle of lateral branches from main trunk: 150-160° degrees.
Branch diameter: 3.5-5 Average 4 cm
Trunk texture: Smooth
Trunk color: Near RHS Brown 200B.
Trunk diameter: Average 7 cm on a 2 year old plant
Foliage:
Leaf:
   Average length.—Typical range, 7-13 cm. Average of 9-9.5 cm.
   Average width.—Typical range, 3.3-6 cm. Average of 4.5-4.6 cm.
   Shape of blade.—Elliptic.
   Apex.—Acute, with very small notch.
   Base.—Broad tapered.
   Attachment.—Petioled.
   Margin.—Entire.
   Texture of top surface.—Glabrous.
   Texture of underside.—Glabrous.
   Color.—Mature foliage upper side: Near RHS Green 137A Mature foliage under side: Near RHS Green 139C.
   Petiole.—Length: Range: 9.0-12.0 mm Average length 9.0-9.5 mm. Width: Average 4 mm Pubescence: None, surfaces glabrous. Color: Upper and lower surfaces near Yellow-Green 147A.
Flower:
Inflorescence and flower type and habit: Single rotate flowers emerging from leaf axils
Flower longevity on plant: approximately 2 weeks.
Persistent or self-cleaning: self-cleaning
Bud:
   Shape.—A small popcorn bud that elongates to a blunt nosed cylindrical bud.
   Length.—Average 8-9 mm.
   Diameter.—Average 5 mm.
   Color.—Near RHS White 155A.
Flower size:
   Diameter.—Average 2.2-2.3 cm.
   Depth.—Average 1.0 cm.
Corolla/petals:
   Arrangement.—Very small.
   Petal quantity.—5.
   Length.—Average 18-10 mm.
   Width.—Average 0.2.5-3 mm.
   Texture.—Smooth without pubescence.
   Apex.—1 mm.
   Base.—2 mm.
   Margin.—Smooth.
Color: Petals are white upon abscission.
Calyx/sepals: Filaments separate easily.
   Quantity per flower.—5.
   Shape.—Straight.
   Length.—Average 6-7 mm.
   Width.—Average 1 mm.
   Texture.—Smooth.
   Color.—Upper Surface: Near RHS Green 137A. Lower Surface: Near RHS Green 137A.
Pedicel:
   Length.—Average 4-5 mm.
   Diameter.—Average 1 mm.
   Color.—Near RHS Green 137A.
   Texture.—Smooth without pubescence.
Fragrance: Sweet, typical Citrus flower scent.
Reproductive organs:
Stamens:
   Number.—Most commonly 12.
   Filament length.—Approximately 2.2 cm.
Anthers:
   Shape.—Globular.
   Length.—Approximately 0.2 cm.
   Color.—Near RHS Yellow-White 158A.
   Pollen.—Color: Near RHS Yellow 8C Quantity: Moderate.
Pistil:
   Number.—1.
   Length.—Approximately 0.8 cm.
   Style.—Length: 0.6 cm. Color: Near RHS Green-Yellow 1D.
   Stigma.—Shape: linear Color: Near RHS Yellow 8D Ovary Color: RHS Green 143C.
Fruit:
General shape: Spherical to very slightly oblate. Not as round as ‘Hamlin’ or ‘Valencia’, but not as oblate as ‘Muvcott’.
Median cross section: 8 cm.
Flesh color when ripe: Between RHS Orange 26A and Orange N25A.
Brix at consumption: 11.60, on the date: Nov. 16, 2011
Brix at harvest: 11.30. Acid 0.68 Ratio of 16.61
Average weight: 230 grams
Minimum weight: 170 grams
Maximum weight: 290 grams
Length: 62-70 mm.
Width: Range: 7-8.5 cm. Average of 8 cm
Segment number per fruit: 9-13
Fruit peduncle length: 80-110 mm
Fruit peduncle width: 60-80 mm.
Skin/rind thickness: 20 mm.
Skin/rind texture: Mostly smooth with very small protruding oil cells.
Skin exterior coloration: Near RHS Orange N25B
Skin interior coloration: Near RHS Yellow-Orange 23C
Harvest time: First Harvest: Early November. Last Harvest: Month of January.
Overall cropping quantity: Above commercial averages.
Mature seed color: Near RHS Yellow-Orange 20D
Quantity seed per fruit: 1-6 seeds per fruits with an average of 3 seeds.
Seed shape: Oval with slight tail.
Seed length: 120-130 mm.
Seed width: 70-80 mm.
Other: A large, low seed fruit that peels extremely easy and dry, with beautiful internal and external color and extremely good taste.
Other characteristics:
Storage life: Storage life is a minimum of 45 plus days at 2° C.
   Disease/pest resistance.—Inventor has not observed resistance nor susceptibility to diseases and pests.
Other fruit features: Does not require cross pollination.

What is claimed is:

1. A new and distinct cultivar of Citrus reticulata plant named ‘C37’ as herein illustrated and described.
